(19) United States
(12) Patent Application Publication
(10) Pub. No.: US 2023/0134935 A1
(43) Pub. Date: May 4, 2023

(54) MEDICAL USE OF DARIDOREXANT

(71) Applicant: Idorsia Pharmaceuticals Ltd, Allschwil (CH)

(72) Inventors: Guy BRAUNSTEIN, Allschwil (CH); Scott PAIN, Allschwil (CH); Dalma SEBOEK-KINTER, Allschwil (CH); Cedric VAILLANT, Allschwil (CH)

(21) Appl. No.: 17/996,499

(22) PCT Filed: Apr. 16, 2021
(86) PCT No.: PCT/EP2021/059943

§ 371 Date: Oct. 18, 2022

(30) Foreign Application Priority Data
Apr. 19, 2020 (WO) PCT/EP2020/060940

(51) Int. Cl. A61K 31/4192; A61P 25/20; A61K 9/28

(52) U.S. Cl. CPC A61K 31/4192; A61P 25/20; A61K 9/2866

(57) ABSTRACT

The invention relates to daridorexant: Formula (I) or drochloric acid salt; for use in a method of treatment of sleep disorders such as especially insomnias, wherein daridorexant improves daytime performance, especially reduces daytime sleepiness associated to such sleep disorder

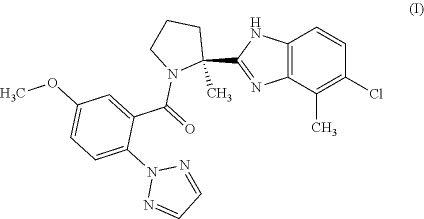 [SMILES: COc1ccc(-n2nccn2)c(C(=O)N2CCC[C@@]2(C)c2nc3c(C)c(Cl)ccc3[nH]2)c1]

### CROSS REFERENCE TO RELATED APPLICATIONS

This application is the National Stage Entry under 35 U.S.C. § 371 of International Application No. PCT/EP2021/059943 filed Apr. 16, 2021, which claims priority to International Application No. PCT/EP2020/060940 filed Apr. 19, 2020, the contents of each of which are hereby incorporated by reference in their entireties.

The present invention relates to the use of daridorexant, [(S)-2-(5-Chloro-4-methyl-1H-benzoimidazol-2-yl)-2-methyl-pyrrolidin-1-yl]-(5-methoxy-2-[1,2,3]triazol-2-yl-phenyl)-methanone (alternatively named ACT-541468):

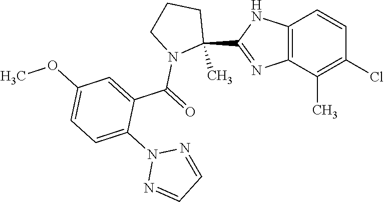 [SMILES: COc1ccc(-n2nccn2)c(C(=O)N2CCC[C@@]2(C)c2nc3c(C)c(Cl)ccc3[nH]2)c1]

or of a pharmaceutically acceptable salt thereof such as especially the hydrochloric acid salt; in the treatment of sleep disorders such as especially insomnias, wherein daridorexant improves daytime performance, especially reduces daytime sleepiness associated to such sleep disorder.

The preparation of daridorexant and the medicinal use thereof is described in WO2013/182972 and WO2015/083094. Crystalline salt forms of daridorexant are disclosed in WO2015/083071; and crystalline forms of the daridorexant in free base form are disclosed in WO2015/083070. In the rat, daridorexant has been shown for example to cross the blood-brain barrier and to promote sleep, characterized by pharmacological effects on active wake, home cage activity, NREM sleep, and REM sleep. Daridorexant has also been reported to be active in an animal model of agitation relevant for sundowning/agitation in dementia (WO2015/083094). In addition, structurally different orexin receptor antagonists have extensively been reported in the literature for pharmacological actions potentially related to mental health diseases or disorders relating to orexinergic dysfunctions such as sleep disorders, anxiety disorders, addiction disorders, cognitive dysfunctions, mood disorders, agitation in dementia, or appetite disorders.

Insomnia (as defined in Diagnostic and Statistical Manual of Mental Disorders, Fifth Edition (DSM-5): American Psychiatric Association. Diagnostic and statistical manual of mental disorders, 5th edition. Arlington, Va.: American Psychiatric Publishing; 2013) is a common problem. Population-based epidemiological studies suggest that 30% or more of the general population complain of sleep disruption and approximately 10% of the general population have complaints of sleep disruption with associated symptoms of distress or daytime functional impairment consistent with the diagnosis of insomnia disorder [NIH Consensus and State-of-the-Science Statements, 2005 June; 13-15; 22(2):1-30; Roth J Clin Sleep Med. 2007; 3 Suppl 5:S7-10]. Insomnia symptoms (difficulties initiating sleep, early-morning awakenings, and dissatisfaction with sleep) increase with age. Factors associated with aging and not with age per se, such as depressed mood, respiratory symptoms, poor perceived health and physical disability are associated with the decrease in ability to sleep [Ohayon et al., J Am Geriatr Soc. 2001; 49(4):360-366]. Insomnia disorder results in difficulty falling asleep or difficulty maintaining sleep, characterized by multiple or long awakenings during the sleep period, or early-morning awakenings. Difficulty maintaining sleep is the most common problem among patients with insomnia, occurring in approximately two-thirds of them [Neubauer, Int Rev Psychiatry. 2014; 26(2):214-24]. Studies have shown that the most common symptoms are combined difficulties to both fall and to stay asleep [Hohagen et al., Sleep. 1994; 17(6):551-4]. Elderly patients are more likely to suffer from chronic insomnia characterized by difficulty maintaining sleep, rather than difficulty initiating sleep [McCall Prim Care Companion J Clin Psychiatry. 2004; 6(1):9-20].

Insomnia is associated with impairment in cognitive functioning, daytime fatigue, increased accident risk, and difficulties in interpersonal relationships. Insomnia increases utilization of medical care, has been correlated with chronic health issues and perceptions of poor health, and in elderly subjects may also precipitate falls [see for example Ancoli-Israel and Roth, Sleep. 1999; 22 Suppl2:S354-58; Zammit et al., Sleep. 1999; 22 Suppl 2:S379-85; Fortier-Brochu and Morin, Sleep. 2014; 37(11):1787-98; McCall Prim Care Companion J Clin Psychiatry. 2004; 6(1):9-20]. Numerous studies have shown an association between insomnia and psychiatric disorders, specifically depression, anxiety, and other significant mental health conditions [Ford and Kamerow, JAMA. 1989; 262(11):1479-84; Benca et al., J Clin Psychiatry. 2004; 65 Suppl 8:26-35].

The current standards of care encompass non-pharmacological therapies and pharmacotherapy [Schutte-Rodin et al., J Clin Sleep Med. 2008; 4:487-504]. Non-pharmacological (psychological and behavioral) standard-of-care therapies for insomnia include a variety of treatment methods, such as cognitive behavioral therapy (CBT), stimulus control and relaxation training. Sleep hygiene therapy is often added to these treatment modalities. Prescription sleep medications (hypnotics) indicated for the treatment of insomnia include benzodiazepines, non-benzodiazepine benzodiazepine receptor agonists, melatonin agonists, the orexin receptor antagonist suvorexant, and low-dose doxepin.

Benzodiazepines are a class of medications that bind to multiple gamma-aminobutyric acid (GABA) type A receptor subtypes [Lieberman Prim Care Companion J Clin Psychiatry. 2007; 9(1):25-31]. Drugs in this class, which includes flurazepam, temazepam, trialozam, estazolam, and quazepam, were previously commonly prescribed for insomnia. While the efficacy of these medications has been well documented, their usefulness is limited by adverse effects such as daytime sedation (e.g., morning or next-day hangover), cognitive impairment (including anterograde amnesia), motor dyscoordination, abuse liability, and dependence [Holbrook et al., CMAJ 2000; 162(2):225-33, Buscemi et al., J Gen Intern Med. 2007; 22(9):1335-50]. Benzodiazepines also alter sleep architecture: they prolong stage 2 sleep and may slightly reduce the relative amount of rapid eye movement (REM) sleep [Drugs for insomnia. Treat Guidel Med Lett. 2009; 7:23-6]. Their use has been associated with tolerance development and rebound insomnia upon withdrawal of medication [Kales et al., Science. 1978; 201(4360):1039-41, Petursson et al., Br J Addict. 1981; 76(2):133-45].

Non-benzodiazepine benzodiazepine receptor agonists have a more targeted action on one or more GABA type A receptor subtypes, but the availability of these treatments highly varies across regions. Zolpidem, zolpidem controlled-release (CR) and zaleplon show affinity for the alpha-1 receptor subtype, while eszopiclone shows affinity for the alpha-2 and -3 receptor subtypes [Nutt J Clin Sleep Med. 2006; 2(2):Suppl S7-11; see also Hair et al., CNS Drugs (2008), 22:975-978]. All of these drugs reduce latency to sleep onset, but zolpidem CR and eszopiclone have also been shown to reduce Wake After Sleep Onset (WASO), reflecting an improvement in sleep maintenance [Ambien® USPI, Lunesta® USPI]. Although they have less impact on sleep architecture, possibly by virtue of their receptor selectivity, the drugs in this group may have have similar adverse effects as the benzodiazepines. In 2007 the US Food and Drug Administration (FDA) requested that all manufacturers of hypnotic drug products strengthen their product labeling to include stronger language related to potential risks. These risks include severe allergic reactions (i.e., anaphylaxis) and complex sleep-related behaviors, which may include sleep-driving [Food and Drug Administration (US) Requests Label Change for All Sleep Disorder Drug Products. Available from: www.fdanews.com/articles/91163-fda-requests-label-change-for-sleep-disorder-drugs]. Nevertheless, hypnotic drugs, including triazolem, zaleplon, zolpidem and eszopiclone, have been proposed as first-line of agents to induce sleep, compared to other classes of agents including e.g. orexin receptor antagonists (suvorexant) (Pagel et al., Sleep Science and Practice (2018): 2-5).

Use of sleep medications increases with age and is highest in the elderly [Ohayon et al., Sleep Med. 2002; 3(2):115-20; Ohayon et al., Sleep Med. 2010; 11(10):1010-8]. Despite the increased risk of falls, non-benzodiazepine benzodiazepine receptor agonists and generic antidepressants are among the most prescribed classes of medications for elderly patients in the USA.

Newer hypnotics that do not act at the GABA receptor have been developed. The melatonin receptor agonist ramelteon is approved for insomnia in the US and in Japan, but not in Europe. Ramelteon reduces sleep latency and increases Total Sleep Time (TST), but has no effect on WASO [Kuriyama et al., Sleep Med. 2014; 15(4):385-92], making it an inappropriate treatment for people with sleep maintenance problems [Simpson and Curran, Drugs. 2008; 68(13):1901-19]. Ramelteon is generally considered devoid of next-day residual effects, withdrawal or rebound insomnia and does not appear to be associated with abuse liability. However, Mets et al. (Sleep (2011), 34 (10): 1327-1334) disclosed a study finding significant next-day residual effects on psychomotor performance, memory, performance, and mood for both ramelteon and zopiclone).

Suvorexant is an oral dual orexin receptor antagonist (DORA) that was approved e.g. in the USA, Japan and Australia for the treatment of insomnia characterized by difficulties with sleep onset and/or sleep maintenance. Suvorexant is contraindicated in patients with narcolepsy. Next-day effects, including impaired driving performance, have been reported at 20 mg [Belsomra® USPI]. Next-day residual effects might be related to the long halflife (t½=12 hours) of suvorexant [Citrome Int J Clin Pract. 2014; 68(12):1429-41]. Rebound insomnia or withdrawal signs upon drug discontinuation were not observed in clinical trials [Herring et al., Biol Psychiatry. 2016; 79(2):136-48]. The label of suvorexant (BELSOMRA®) in the USA includes the warnings and precautions typical for drugs indicated for the treatment of insomnias: “CNS Depressant Effects and Daytime Impairment: Risk of impaired alertness and motor coordination, including impaired driving; risk increases with dose; caution patients taking 20 mg against next-day driving and other activities requiring complete mental alertness”, and in more detail: “BELSOMRA® is a central nervous system (CNS) depressant that can impair daytime wakefulness even when used as prescribed.”

Similarly, lemborexant (DAYVIGO®) another DORA was approved in the US with a label that includes similar warnings and precautions: “CNS Depressant Effects and Daytime Impairment: Impairs alertness and motor coordination including morning impairment. Risk increases with dose and use with other central nervous system (CNS) depressants. For patients taking DAYVIGO® 10 mg, caution against next-day driving and other activities requiring complete mental alertness.” WO2016/063995/U.S. Pat. No. 10,188,652 claims “A method of treating insomnia, comprising administrating orally a dosage form comprising a therapeutically effective amount of [lemborexant], wherein said therapeutically effective amount is a single daily dose ranging from about 2.5 mg to about 10 mg, wherein said single daily dose achieves a mean Cmax of from about 3.0 ng/ml to about 7.2 ng/ml for each 1 mg of [lemborexant] after administration to human subjects.” In the prosecution of the application at the USPTO it was stated that “[ . . . ] the present application contains data showing that the single daily dosage ranging from about 2.5 mg to about 10 mg, administered orally in humans, provides rapid sleep onset without leading to next day sleepiness or impairment. See Examples 3-5. In fact, a dose of 2.5 mg or more of [lemborexant] was needed to effectively induce sleep onset (see para [0242]), and at dosages greater than 10 mg, patients demonstrated a greater increase in next-day sleepiness (see paragraph [0244]).” Thus, an increasing dose of lemborexant may be assumed (at >10 mg) to lead to a greater increase of next-day sleepiness, and a narrow window of dosages between 2.5 mg and 10 mg single dose per day needs to be selected where the compound is effectively inducing sleep onset without leading to next day sleepiness or impairment.

CNS Depressant Effects and Daytime Impairment warnings and precautions result from an overall evaluation of the respective health authority, generally taking into consideration outcomes of clinical studies in patients as well as dedicated safety studies in healthy volunteers; which studies (e.g. clinical phase 2 studies or safety studies) may include investigation of supra-therapeutic doses (i.e. higher doses than finally approved by the respective health authority for treatment).

Clinical phase 3 studies of suvorexant (NCT01097616, NCT01097629) and lemborexant (NCT02783729, NCT02952820) in insomnia patients observed on exploratory basis (i.e. not in form of a validated clinical endpoint) in post-hoc analyses the effects of compound treatment on the Insomnia Severity Score (ISI©) [Herring et al., Sleep Medicine 56 (2019) 219-223: doi.org/10.1016/j.sleep.2018.09.010; Rosenberg et al., JAMA Network Open. 2019; 2(12):e1918254. doi:10.1001/jamanetworkopen.2019.18254]. The Insomnia Severity Index (ISI©) (Morin et al.; SLEEP 2011; 34(5):601-608), in its entirety, is a validated brief instrument that was designed to assess the severity of both nighttime and daytime components of insomnia assesses the severity of a patient's insomnia by scoring the severity of sleep onset and sleep maintenance difficulties and any insomnia-related interference with daytime functioning. The assessment is on a 5-point scale (0-4), where the composite score is obtained by summing the 7 rated dimensions measuring the subject's perception of his or her insomnia. A score of 15-21 indicates a moderate level of insomnia and a score of 22-28 indicates severe insomnia. An ISI© total score <10 indicates that the subject's subjectively-rated insomnia symptoms, daytime impairment, and quality of life have improved to the minimal-to-none range [Morin et al., J Consult Clin Psychol. 1993; 61(1):137-46, Scharf et al., Sleep. 2007; 30(6):743-52; Morin et al.; SLEEP 2011; 34(5):601-608].

Lemborexant was further evaluated on exploratory basis in a more daytime related ISI score, i.e. a subset pooling scores of only items 4 to 7 (Roth et al, Poster presented at the 33rd Annual Meeting of the Associated Professional Sleep Societies (APSS); Jun. 8-12, 2019; San Antonio, Tex.). ISI© items 4 to 7 evaluate the following questions:

4. How SATISFIED/DISSATISFIED are you with your CURRENT sleep pattern?

5. How NOTICEABLE to others do you think your sleep problem is in terms of impairing the quality of your life?

6. How WORRIED/DISTRESSED are you about your current sleep problem?

7. To what extent do you consider your sleep problem to INTERFERE with your daily functioning (e.g. daytime fatigue, mood, ability to function at work/daily chores, concentration, memory, mood, etc.) CURRENTLY?

Both lemborexant and suvorexant were not assessed for a validated clinical endpoint related to daytime impairment/daytime sleepiness in insomnia patients. Furthermore, the ISI© generally has a recall period of one month, thus, not allowing measurement of the day-to-day variability of daytime impairment, and only item 7 of the ISI© as set out above is specifically directed to daytime functioning. However, the ISI© instrument is not designed to provide meaningful results for particular item sub-sets. For example, the above mentioned post-hoc analysis for lemborexant presents results of a pooled outcome for sub-set ISI© items 4 to 7. Thus, the ISI© may not be considered as a validated tool to assess daytime performance/daytime functioning.

Insomnia disorder is a chronic disease and currently available treatments are generally limited to short-term use with the exception of eszopiclone, suvorexant, and lemborexant. Caution and dose reduction are also often advised in the elderly. Pharmacological treatments that address sleep onset problems alone do not provide relief to people with sleep maintenance difficulties, and treatments indicated for those with sleep maintenance problems may be associated with risks of cognitive impairment, postural instability, or next-day residual sedation that may impair driving [Neubauer Int Rev Psychiatry. 2014; 26(2):214-24].

Moreover, the use of benzodiazepines and benzodiazepine receptor agonists is associated with an increased risk of falling [McCall Prim Care Companion J Clin Psychiatry. 2004; 6(1):9-20] leading to hip and femur fractures, increased disability, and use of healthcare resources. In September 2020 an FDA Drug Safety Communication (www.fda.gov/drugs/drug-safety-and-availability/fda-requiring-boxed-warning-updated-improve-safe-use-benzodiazepine-drug-class) was issued requiring a Boxed Warning updated to improve safe use of benzodiazepine drug class includes potential for abuse, addiction, and other serious risks:
“To address the serious risks of abuse, addiction, physical dependence, and withdrawal reactions, the U.S. Food and Drug Administration (FDA) is requiring the Boxed Warning be updated for all benzodiazepine medicines. Benzodiazepines are widely used to treat many conditions, including anxiety, insomnia, and seizures. The current prescribing information for benzodiazepines does not provide adequate warnings about these serious risks and harms associated with these medicines so they may be prescribed and used inappropriately. This increases these serious risks, especially when benzodiazepines are used with some other medicines and substances.
Benzodiazepines can be an important treatment option for treating disorders for which these drugs are indicated. However, even when taken at recommended dosages, their use can lead to misuse, abuse, and addiction. Abuse and misuse can result in overdose or death, especially when benzodiazepines are combined with other medicines, such as opioid pain relievers, alcohol, or illicit drugs. Physical dependence can occur when benzodiazepines are taken steadily for several days to weeks, even as prescribed. Stopping them abruptly or reducing the dosage too quickly can result in withdrawal reactions, including seizures, which can be life-threatening.”

Overall, there is a need for a long-term (chronic) pharmacological treatment for insomnia disorder that addresses the most prominent and pressing symptoms/clinical manifestations of insomnia without negatively impacting next-day functioning. The latter being a key criterion in defining insomnia as per DSM-5 i.e., the sleep disturbance causes clinically significant distress or impairment in social, occupational, educational, academic, behavioral, or other important areas of functioning [American Psychiatric Association. Diagnostic and statistical manual of mental disorders, 5th edition. Arlington, Va.: American Psychiatric Publishing; 2013].

Daridorexant has been evaluated in a phase 2 clinical trial (NCT02839200; Dauvilliers et al, Ann Neurol 2020; 87:347-356; Idorsia media release Jun. 13, 2019) and was found to induce a dose-dependent reduction in wake time after sleep onset in subjects with insomnia disorder. Human simulations of daridorexant suggested high and rapid peak orexin receptor occupancy and fast decline in receptor occupancy at a dose of 25 mg. In the phase 2 clinical trial, “visual analog scales (VASs) on morning sleepiness, daytime alertness, and daytime ability to function all showed nonsignificant increases in a dose-dependent manner at higher doses of daridorexant compared with placebo at week 2, which was not sustained at week 4. This pattern was also true for zolpidem compared with placebo.” The authors conclude that “a difference in half-life of several hours can have an important impact on the propensity to elicit next-morning residual drug effects, emphasized in the current study by the absence of such effects up to and including the highest dose (i.e. 50 mg)”. The authors further state that “one of the main concerns with current insomnia products is the potential for residual next-morning effects. The assessment of morning sleepiness showed no difference between any dose of daridorexant and placebo in terms of sleepiness the following day. This is assumed to be related to the short half-life of daridorexant, as shown in phase 1 clinical trials in healthy subjects. The relatively short half-life of daridorexant may also result in fewer residual effects. In addition, daridorexant, in common with all DORAs, is designed to improve the quality of sleep, so an absence of next-morning residual effects would not be unexpected and would support the data generated showing objective improvement.”

Daridorexant entered two phase 3 multi-center, double-blind, randomized, placebo-controlled, parallel-group, polysomnography study to assess the efficacy and safety of ACT-541468 in adult and elderly subjects with insomnia disorder. (NCT03545191, EudraCT Number: 2017-004642-20 testing strengths of 25 mg and 50 mg; and NCT03575104, EudraCT Number: 2017-004643-20 testing strengths of 10 mg and 25 mg). To assess daytime functioning, said phase 3 studies investigate as a secondary endpoint the IDSIQ sleepiness domain score, using as a dedicated patient reported outcome (PRO) instrument the Insomnia Daytime Symptoms and Impacts Questionnaire (IDSIQ) (S. Hudgens et al., The Patient—Patient-Centered Outcomes Research (2021) 14:249-268). The instrument is based on the Daytime Insomnia Symptom Scale (DISS) (Buysse et al., Sleep Medicine 8 (2007) 198-208; Buysse et al., SLEEP 29(9) (2006) 1155-1173) and designed to characterize and compare daytime symptoms in insomnia. The IDSIQ is structured in three domains (alertness/cognition; (negative) mood; tiredness/sleepiness) and contains overall 14 items, each based on an 11-point numeric rating scale. For each domain score the total rating score of all associated items is considered.

The daytime alertness/cognition domain score comprises the six items:
How clear-headed did you feel today? 11-point numeric rating scale from—not at all clear-headed to—very clear-headed.
How well were you able to concentrate today? 11-point numeric rating scale from—not able to concentrate at all to—able to concentrate very well.
How forgetful did you feel today? 11-point numeric rating scale from—not at all forgetful to—very forgetful.
How much of an effort was it to perform daily activities (i.e., reading, cleaning, work, school) today? 11-point numeric rating scale from no effort at all to a lot of effort.
How refreshed did you feel today? 11-point numeric rating scale from—not at all refreshed to—very refreshed.
How awake did you feel today? 11-point numeric rating scale from—not at all awake to—very awake.

The daytime (negative) mood domain score comprises the four items:
How worried did you feel today? 11-point numeric rating scale from not at all worried to very worried.
How frustrated by your lack of sleep did you feel today? 11-point numeric rating scale from—not at all frustrated to—very frustrated.
How irritable did you feel today? 11-point numeric rating scale from—not at all irritable to—very irritable.
How stressed did you feel today? 11-point numeric rating scale from—not at all stressed to—very stressed.

The daytime sleepiness domain score (which may alternatively be named for example daytime sleepiness/tiredness domain score) comprises the four items:
How energetic did you feel today? 11-point numeric rating scale from—not at all energetic to—very energetic.
How mentally tired did you feel today? 11-point numeric rating scale from—not at all mentally tired to—very mentally tired.
How physically tired did you feel today? 11-point numeric rating scale from—not at all physically tired to—very physically tired.
How sleepy did you feel today? 11-point numeric rating scale from not at all sleepy to very sleepy.

It has now been found that daridorexant in clinical phase 3 study NCT03545191 confirmed prior clinical phase 2 data and significantly improved sleep onset [with significant reductions in latency to persistent sleep (LPS)] and sleep maintenance [with significant reductions in wake time after sleep onset (WASO)] at doses of 25 mg and 50 mg; and increased the subjective total sleep time (sTST) significantly at doses of 25 mg and 50 mg. Clinical phase 3 study NCT03575104, EudraCT Number: 2017-004643-20 showed that daridorexant 25 mg, but not 10 mg, significantly decreased WASO and increased sTST at month 1 and month 3 versus placebo, while the decrease in LPS did not reach statistical significance (significant after log-transformation) in this trial. Surprisingly, daridorexant, in addition to the clinical effects in the night (i.e. directly on sleep parameters such as sleep onset and/or maintenance as assessed by WASO, LPS and/or sTST), also improved the next day performance of the patients, especially as measured as secondary clinical endpoint using the IDSIQ, in particular the IDSIQ sleepiness domain score. Daridorexant, while showing numerical improvement at the lower dose of 25 mg, significantly improved (reduced) the IDSIQ sleepiness domain score versus placebo at the higher dose of 50 mg. In addition, daridorexant improved IDSIQ mood and alert/cognition domains and total score at month 1 and month 3. The data show that improvements in sleep (WASO, LPS, sTST) are maintained over three months and are associated with a gradual improvement in daytime functioning over time.

### DESCRIPTION OF THE FIGURES

FIG. 1: Primary and secondary efficacy end points

FIG. 2: Other IDSIQ end points.

### DETAILED DESCRIPTION OF THE INVENTION

1) A first embodiment relates to a method of treating a sleep disorder (especially an insomnia); said method comprising administering to a subject in need thereof a pharmacologically effective amount of daridorexant, or of a pharmaceutically acceptable salt thereof; wherein daridorexant improves the daytime performance of said subject.

It is understood that the term “treatment of a sleep disorder” in particular comprises the aspects sleep onset [e.g. as assessed by latency to persistent sleep (LPS)] and/or sleep maintenance [e.g. as assessed by wake time after sleep onset (WASO)] and/or subjective total sleep time (sTST).

It is further understood that such improvement in daytime performance may preferably be assessed by the Insomnia Daytime Symptoms and Impacts Questionnaire (IDSIQ) patient reported outcome instrument. In particular, the total IDSIQ score is defined as comprising:
the IDSIQ daytime alert/cognition domain score,
the IDSIQ daytime mood domain score, and
the IDSIQ daytime sleepiness domain score.

The term “daytime performance” is understood to be equivalent to the term “daytime functioning” and the terms may be used interchangeably herein.

The daytime alertness/cognition domain score comprises the six items:
How clear-headed did you feel today? 11-point numeric rating scale from—not at all clear-headed to—very clear-headed.
How well were you able to concentrate today? 11-point numeric rating scale from—not able to concentrate at all to—able to concentrate very well.
How forgetful did you feel today? 11-point numeric rating scale from—not at all forgetful to—very forgetful.
How much of an effort was it to perform daily activities (i.e., reading, cleaning, work, school) today? 11-point numeric rating scale from no effort at all to a lot of effort.
How refreshed did you feel today? 11-point numeric rating scale from—not at all refreshed to—very refreshed.
How awake did you feel today? 11-point numeric rating scale from—not at all awake to—very awake.

The daytime (negative) mood domain score comprises the four items:
How worried did you feel today? 11-point numeric rating scale from not at all worried to very worried.
How frustrated by your lack of sleep did you feel today? 11-point numeric rating scale from—not at all frustrated to—very frustrated.
How irritable did you feel today? 11-point numeric rating scale from—not at all irritable to—very irritable.
How stressed did you feel today? 11-point numeric rating scale from—not at all stressed to—very stressed.

The daytime sleepiness domain score comprises the four items:
How energetic did you feel today? 11-point numeric rating scale from—not at all energetic to—very energetic.
How mentally tired did you feel today? 11-point numeric rating scale from—not at all mentally tired to—very mentally tired.
How physically tired did you feel today? 11-point numeric rating scale from—not at all physically tired to—very physically tired.
How sleepy did you feel today? 11-point numeric rating scale from not at all sleepy to very sleepy.

For avoidance of doubt, any reference herein to the active ingredient daridorexant in free or pharmaceutically acceptable salt form is understood to refer interchangeably to daridorexant, or a pharmaceutically acceptable salt thereof; such pharmaceutically acceptable salt form of daridorexant being especially the hydrochloric acid salt form.

2) A further embodiment, thus, relates to the method according to embodiment 1); wherein said improvement of daytime performance of said subject is assessed by at least one of:
the IDSIQ daytime alert/cognition domain score; and/or
the IDSIQ daytime mood domain score, and/or
the IDSIQ daytime sleepiness domain score;

wherein each IDSIQ daytime domain score, i.e. the alert/cognition domain score, the daytime mood domain score, and the daytime sleepiness domain score (each as defiend before), each forms a separate sub-embodiment.

In as sub-embodiment, a particular aspect of the invention relates to such improvement of daytime performance according to embodiment 1) or 2), and mutatis mutandis any one of embodiments 3) to 54) herein below, wherein said daytime performance improves over time, i.e. the amplitude of effect on daytime performance/daytime functioning increases over time of treatment (e.g. during at least 4 weeks or at least 12 weeks such as especially from week 1 (through week 4) to at least week 12 of treatment).

3) A further embodiment relates to the method according to embodiment 1); wherein said improvement of daytime performance of said subject is assessed by the IDSIQ daytime sleepiness domain score.

4) Another aspect of the invention relates to a method of treating a sleep disorder (especially an insomnia); said method comprising administering to a subject in need thereof a pharmacologically effective amount of daridorexant, or of a pharmaceutically acceptable salt thereof; wherein daridorexant reduces daytime clinical manifestations associated with said sleep disorder (especially insomnia).

It is understood that such reduction of daytime clinical manifestations may especially be assessed by the Insomnia Daytime Symptoms and Impacts Questionnaire (IDSIQ) patient reported outcome instrument; wherein mutatis mutandis embodiments 2) and 3) apply.

5) A further embodiment relates to the method according to embodiment 4); wherein said daytime clinical manifestations associated with said sleep disorder are symptoms of daytime impairment.

6) A further embodiment relates to the method according to embodiment 4); wherein said daytime clinical manifestations associated with said sleep disorder are symptoms of daytime sleepiness, especially as assessed by the IDSIQ daytime sleepiness domain score.

7) A further embodiment relates to the method according to any one of embodiment 1) to 6); wherein said improvement of daytime performance/reduction of daytime clinical manifestations associated with said sleep disorder is expressed by subjects to whom daridorexant has been administered, wherein said subjects feel, cumulatively, less physically tired, less mentally tired, less sleepy and more energetic during the day.

8) A further embodiment relates to the method according to any one of embodiment 1) to 7); wherein said sleep disorder is a dyssomnia or a sleep disorder associated with a general medical condition.

9) A further embodiment relates to the method according to any one of embodiment 1) to 7); wherein said sleep disorder is a dyssomnia.

10) A further embodiment relates to the method according to any one of embodiment 1) to 7); wherein said sleep disorder is a sleep disorder associated with a general medical condition.

11) A further embodiment relates to the method according to any one of embodiment 1) to 7); wherein said sleep disorder is an insomnia associated with a mood disorder (especially a depressive disorder), an insomnia associated with epilepsy, an insomnia associated with an autism spectrum disorder, an insomnia associated with attention deficit hyperactivity disorder (ADHD), an insomnia associated with a cerebral neurodegenerative disorder (especially Alzheimer's disease); an insomnia associated with an anxiety disorder, an insomnia associated with an addiction disorder, or an insomnia associated with an appetite disorder.

12) A further embodiment relates to the method according to any one of embodiment 1) to 10); wherein said sleep disorder is an insomnia.

13) A further embodiment relates to the method according to any one of embodiment 1) to 9); wherein said sleep disorder is primary insomnia.

14) A further embodiment relates to the method according to any one of embodiment 1) to 13); wherein said treatment of said sleep disorder (especially insomnia) results in at least one, preferably all of the following treatment effects:
an improvement on sleep onset/a decrease in latency to persistent sleep (LPS); and/or
an improvement on sleep maintenance/a decrease in wake time after sleep onset (WASO); and/or
an increase in total sleep time, e.g. as subjectively assessed daily by the patient (sTST); wherein it is understood that the above effects especially are measured from baseline and compared to placebo; wherein it is understood that said treatment effects especially are statistically significant.

15) A further embodiment relates to the method according to any one of embodiment 1) to 13); wherein said treatment of said sleep disorder (especially insomnia) results in the following treatment effects:
an improvement on sleep onset/a decrease in latency to persistent sleep (LPS); and
an improvement on sleep maintenance/a decrease in wake time after sleep onset (WASO);

wherein it is understood that the above effects especially are measured from baseline and compared to placebo; wherein it is understood that said treatment effects especially are statistically significant.

16) A further embodiment relates to the method according to any one of embodiment 1) to 13); wherein in said treatment of said sleep disorder (especially insomnia) results in the following treatment effects:
an improvement on sleep onset/a decrease in latency to persistent sleep (LPS); and
an improvement on sleep maintenance/a decrease in wake time after sleep onset (WASO); and
an increase in total sleep time subjectively (sTST) assessed daily by the patient;

wherein it is understood that the above effects especially are measured from baseline and compared to placebo; wherein it is understood that said treatment effects especially are statistically significant.

17) A further embodiment relates to the method according to any one of embodiment 1) to 16); wherein said subject is an adult.

18) A further embodiment relates to the method according to any one of embodiment 1) to 16); wherein said subject is an elderly adult (defined as being 65 or older).

19) A further embodiment relates to the method according to any one of embodiment 1) to 16); wherein said subject is a pediatric patient (defined as being younger than 18). It is understood that the dose of daridorexant in free or pharmaceutically acceptable salt form (as set out in any one of embodiments 21) to 24) below may need to be adapted to the pediatric population.

Any unit dose/dosage may need to be adapted to the body weight e.g. in the pediatric population. Generally, in pediatric patients a weight scaling may be required. For daridorexant, weight scaling may especially be required for example in (pediatric) patients having a body weight of less than 40 kg. Such adapted/reduced doses are equivalent to the respective dose for the adult patient population (such as a unit dose of about 25 mg of daridorexant in free base equivalent, or a unit dose of about 50 mg of daridorexant in free base equivalent); and shall be encompassed in the respective adult unit dose/dosage.

20) A further embodiment relates to the method according to any one of embodiment 1) to 19); wherein said subject has been diagnosed as having difficulties with sleep onset and/or sleep maintenance.

21) Another aspect of the invention relates to the method of any one of embodiments 1) to 20), wherein daridorexant, or a pharmaceutically acceptable salt thereof, is administered in a unit dose of about 25 mg to about 50 mg of daridorexant; in particular in a unit dose of about 25 mg of daridorexant or in a unit dose of about 50 mg of daridorexant (wherein it is understood that the amount of daridorexant in salt form is given as the free base equivalent).

It is understood that the amount of daridorexant in said unit dose of daridorexant of embodiment 21) [and, likewise, embodiments 22) to 24) below] is given as the free base equivalent. When adminstered in pharmaceutically acceptable salt form the amount refers to daridorexant in free base form (i.e. refers to the amount of daridorexant given as the free base equivalent) and the actual amount of daridorexant in such pharmaceutically acceptable salt form (such as especially daridorexant in hydrochloric acid salt form) may need to be adapted. For example, a unit dose of about 25 mg of daridorexant active ingredient given as the free base equivalent corresponds to unit dose of about 27 mg of daridorexant in hydrochloric acid salt form; and a unit dose of about 50 mg of daridorexant active ingredient given as the free base equivalent corresponds to about 54 mg of daridorexant in hydrochloric acid salt form. Herein, any reference to a unit dose is given as the amount of daridorexant active ingredient as the free base equivalent.

22) A further embodiment relates to the method of any one of embodiments 1) to 20), wherein daridorexant, or a pharmaceutically acceptable salt thereof, is administered in a unit dose of about 25 mg of daridorexant, or in a unit dose of about 50 mg of daridorexant (wherein it is understood that the amount of daridorexant in salt form is given herein as the free base equivalent).

23) A further embodiment relates to the method of any one of embodiments 1) to 20), wherein daridorexant, or a pharmaceutically acceptable salt thereof, is administered in a unit dose of about 25 mg of daridorexant (wherein it is understood that the amount of daridorexant in salt form is given herein as the free base equivalent).

24) A further embodiment relates to the method of any one of embodiments 1) to 20), wherein daridorexant, or a pharmaceutically acceptable salt thereof, is administered in a unit dose of about 50 mg of daridorexant (wherein it is understood that the amount of daridorexant in salt form is given as the free base equivalent).

25) A further embodiment relates to the method of any one of embodiments 1) to 24), wherein the daridorexant is in hydrochloric acid salt form.

26) A further embodiment relates to the method of any one of embodiments 1) to 24), or mutatis mutandis 25), wherein the daridorexant (i.e. daridorexant in free or pharmaceutically acceptable salt form, especially daridorexant in hydrochloric acid salt form) is administered in form of a tablet.

27) A further embodiment relates to the method of any one of embodiments 1) to 24), or mutatis mutandis 25), wherein the daridorexant (i.e. daridorexant in free or pharmaceutically acceptable salt form, especially daridorexant in hydrochloric acid salt form) is administered in form of a tablet; wherein said tablet is a film coated tablet comprising
a tablet core; said tablet core comprising at least two, preferably all of the following excipients: mannitol, microcrystalline cellulose, povidone, croscarmellose sodium, silicon dioxide, and/or magnesium stearate; and
a film coat; said film coat comprising at least two, preferably all of the following excipients: hypromellose, microcrystalline cellulose, glycerine, talc, titanium dioxide, and/or iron oxide.

28) A further embodiment relates to the method of any one of embodiments 1) to 27), wherein daridorexant (i.e. daridorexant in free or pharmaceutically acceptable salt form, especially daridorexant in hydrochloric acid salt form) is administered once daily.

29) A further embodiment relates to the method of any one of embodiments 1) to 28), wherein daridorexant (i.e. daridorexant in free or pharmaceutically acceptable salt form, especially daridorexant in hydrochloric acid salt form) is administered in the evening.

30) A further embodiment relates to the method of any one of embodiments 1) to 28), wherein daridorexant (i.e. daridorexant in free or pharmaceutically acceptable salt form, especially daridorexant in hydrochloric acid salt form) is administered within 2 h before bedtime, especially within 1 h before bedtime, in particular within 30 min before bedtime.

31) A further embodiment relates to the method of any one of embodiments 1) to 28), wherein daridorexant (i.e. daridorexant in free or pharmaceutically acceptable salt form, especially daridorexant in hydrochloric acid salt form) is administered within 1 h before bedtime, in particular within 30 min before bedtime. In a sub-embodiment, said unit dose of daridorexant is administered just before bedtime.

32) A further embodiment relates to the method of any one of embodiments 1) to 28), wherein daridorexant (i.e. daridorexant in free or pharmaceutically acceptable salt form, especially daridorexant in hydrochloric acid salt form) is administered within between about 0.25 h to 2 h (in particular between about 0.25 h to 1 h) before bedtime. In a sub-embodiment, said unit dose of daridorexant is administered just before bedtime.

33) A further embodiment relates to the method according to any one of embodiment 1) to 32); wherein the subject exhibits a significant reduction from baseline in sleep maintenance, notably as measured by WASO, in particular after one month and/or three months of treatment.

34) A further embodiment relates to the method according to any one of embodiment 1) to 32); wherein the subject exhibits a significant reduction from baseline in sleep maintenance, notably as measured by WASO, in particular after three months of treatment.

35) A further embodiment relates to the method according to any one of embodiment 1) to 34); wherein the subject exhibits a significant reduction from baseline in sleep onset, notably as measured by LPS, in particular after one month and/or three months of treatment.

36) A further embodiment relates to the method according to any one of embodiment 1) to 34); wherein the subject exhibits a significant reduction from baseline in sleep onset, notably as measured by LPS, in particular after three months of treatment.

37) A further embodiment relates to the method according to any one of embodiment 1) to 36); wherein the subject exhibits a significant increase from baseline in total sleep time, notably as measured by sTST, in particular after one month and/or three months of treatment.

38) A further embodiment relates to the method according to any one of embodiment 1) to 36); wherein the subject exhibits a significant increase from baseline in total sleep time, notably as measured by sTST, in particular after three months of treatment.

39) A further embodiment relates to the method according to any one of embodiment 1) to 38); wherein said sleep disorder is a chronic sleep disorder.

40) A further embodiment relates to the method according to any one of embodiment 1) to 39); wherein the duration of treatment is at least three months.

41) A further embodiment relates to the method according to any one of embodiment 1) to 39); wherein the duration of treatment is at least nine months.

42) A further embodiment relates to the method according to any one of embodiment 1) to 41); wherein said sleep disorder is caused by or consequent to withdrawal from benzodiazepines or from non-benzodiazepine benzodiazepine receptor agonists.

43) A further embodiment relates to the method according to any one of embodiment 1) to 42); wherein the subject suffers from anxiety/has been diagnosed as having an anxiety disorder.

44) A further embodiment relates to the method according to any one of embodiment 1) to 43); wherein the sleep disorder is insomnia characterized by difficulties with sleep onset and/or sleep maintenance.

45) A further embodiment relates to the method according to any one of embodiment 1) to 44); wherein the daridorexant in free or pharmaceutically acceptable salt form is taken no more than once per night, notably within 30 minutes of going to bed, and especially with at least 7 hours remaining before the planned time of awakening.

46) Another aspect of the present invention relates to a method of treating daytime sleepiness in a subject, comprising administering to said subject an effective amount of daridorexant, or of a pharmaceutically acceptable salt thereof (in particular daridorexant in hydrochloric acid salt form), wherein said subject has a reduction in daytime sleepiness as assessed by the IDSIQ daytime sleepiness domain score; wherein especially said subject has been diagnosed as having a sleep disorder.

47) A further embodiment relates to the method of embodiment 46) wherein one or more of the characteristics of any one of embodiments 7) to 32) apply mutatis mutandis.

48) A further embodiment relates to the method of embodiment 46) or 47), wherein one or more of the characteristics of any one of embodiments 33) to 45) apply mutatis mutandis.

49) Another aspect of the present invention relates to a method of treating insomnia to improve sleep and daytime functioning, comprising administering to a subject in need thereof an effective amount of daridorexant in free or pharmaceutically acceptable salt form (in particular daridorexant in hydrochloric acid salt form), wherein notably said subject has a reduction in daytime sleepiness as assessed by the IDSIQ daytime sleepiness domain score.

50) A further embodiment relates to the method of embodiment 49), wherein said subject is a patient with sleep onset and/or sleep maintenance difficulties (wherein especially said subject is an adult patient).

51) A further embodiment relates to the method of embodiment 49) or 50), wherein one or more of the characteristics of any one of embodiments 1) to 32) apply mutatis mutandis.

52) A further embodiment relates to the method of embodiment 49) or 50), wherein one or more of the characteristics of any one of embodiments 33) to 45) apply mutatis mutandis.

53) A further embodiment relates to daridorexant, or a pharmaceutically acceptable salt thereof, for use in a method according to any one of embodiments 1) to 32).

54) A further embodiment relates to daridorexant, or a pharmaceutically acceptable salt thereof, for use in a method according to any one of embodiments 33) to 52).

For avoidance of doubt, for the present invention any amount/unit dose of daridorexant refers to the amount/unit dose suitable for the administration of daridorexant in free base form in such amount/unit dose. Such amount/unit dose may need to be adjusted in a pharmaceutical composition in case daridorexant is present in such composition in a form different from anhydrous free base, such as a in form of a pharmaceutically acceptable salt, e.g. the hydrochloric acid salt; and/or a solvate such as a hydrate.

Whenever a certain dosage refers to a unit dose of a certain amount in mg, it is understood that such unit dose refers to such amount in mg of daridorexant active ingredient in free base form having a molecular weight of 450.93 g/mol. In case the active ingredient is administered e.g. in form of a pharmaceutically acceptable salt such as the hydrochloric acid salt form, it is understood that the respective amount of active pharmaceutical ingredient (e.g. said pharmaceutically acceptable salt) in a pharmaceutical composition will be adapted accordingly (e.g. 487.39 g/mol for the hydrochloric acid salt, thus, for example a unit dose of about 27 mg of daridorexant HCl corresponds to a unit dose of about 25 mg of daridorexant active ingredient; and about 54 mg of daridorexant HCl corresponds to a unit dose of about 50 mg of daridorexant active ingredient).

An effective amount is preferably to be understood as pharmacologically effective amount.

Where the plural form is used for compounds, solid, pharmaceutical compositions, diseases and the like, this is intended to mean also a single compound, solid, or the like.

The term “consisting essentially of” is understood in the context of the present invention to mean especially that the respective composition consists in an amount of at least 90, notably of at least 95, especially of at least 99, and preferably in an amount of 100 percent by weight (i.e. in the meaning of “consisting of”) of the respective composition in the amounts as explicitly stated in the respective embodiment. The term “comprising” is preferably to be understood in the meaning of the term “consisting essentially of”.

The term “essentially”, for example when used in a term such as “essentially pure” is understood in the context of the present invention to mean especially that the respective composition/compound etc. consists in an amount of at least 90, especially of at least 95, and notably of at least 99 percent by weight of the respective pure composition/compound/crystalline form etc.

The term “enantiomerically enriched” is understood in the context of the present invention to mean especially that at least 90, preferably at least 95, and most preferably at least 99 percent by weight of daridorexant are present in form of one enantiomer of daridorexant. It is understood that daridorexant is present in enantiomerically enriched absolute (S)-configuration.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C., notably to an interval extending from Y minus 3° C. to Y plus 3° C. Room temperature means a temperature of about 25° C. When in the current application the term n equivalent(s) is used wherein n is a number, it is meant and within the scope of the current application that n is referring to about the number n, preferably n is referring to the exact number n.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or from 1 to 4), this means that the variable is the integer 1, 2, 3, or 4.

Daridorexant can be used as medicament according to this invention, e.g. in the form of pharmaceutical compositions especially for enteral, or for parenteral administration.

For avoidance of any doubt, it is understood that any pharmaceutical composition comprising daridorexant in a pharmaceutically effective amount may additionally comprise further conventional excipients and/or additives, which may be used alone or in combination (quantum satis, i.e. wherein the maximum amounts of said further conventional ingredients and/or additives may need to be reduced to make up the total ww % of 100). It is understood that the total amount expressed in “ww %” of a certain composition is 100.

Reference is made to the extensive literature on the subject for these and other pharmaceutically acceptable excipients and procedures mentioned herein, see for example R. C. Rowe, P. J. Seskey, S. C. Owen, Handbook of Pharmaceutical Excipients, 5th edition, Pharmaceutical Press 2006; Remington, The Science and Practice of Pharmacy, 21st Edition (2005), Part 5, “Pharmaceutical Manufacturing” [published by Lippincott Williams & Wilkins].

The expression “ww %” (or % (w/w)) refers to a percentage by weight compared to the total weight of the composition considered. If not explicitly stated otherwise, the considered total weight is the total weight of the pharmaceutical composition.

The expression (wt/wt) relating to a ratio refers to a ratio by weight of the respective components.

In case a certain value is given as % value, in absence of further specification such value refers to ww %, or if in the context of purity, area % as measured by HPLC.

Dosage forms suitable for enteral administration may be tablets or capsules (especially tablets) comprising a pharmaceutical composition comprising an efficacious amount of daridorexant.

The term “pharmaceutical composition” is interchangeable with the terms “formulation”, or “composition”.

The term “treat” or “treatment” or “treating” used with reference to a disease means either that said disease is cured in the patient or animal; or that, although the animal or patient remains affected by the disease, part or all of the symptoms of said disease are either reduced or eliminated.

The terms “subject”, and likewise, “patient” refers to mammals, especially humans. Notably, the term “subject” refers to a human patient.

Sleep disorders comprise especially dyssomnias and sleep disorders associated with a general medical condition, as well as parasomnias, and substance-induced sleep disorders. Dyssomnias in particular include intrinsic sleep disorders (especially insomnias, breathing-related sleep disorders, periodic limb movement disorder, and restless leg syndrome), extrinsic sleep disorders, and circadian-rythm sleep disorders. Sleep disorders notably refer to all types of insomnia including primary insomnia and idiopathic insomnia; intermittent treatment of chronic insomnia; situational transient insomnia (e.g. insomnia associated to a new environment or noise); (short-term) insomnia due to stress, grief, pain or illness; and insomnias related to mental or neurologic disorders including insomnias associated with mood disorders (such as depressive disorders), epilepsy, autism spectrum disorders, attention deficit hyperactivity disorder (ADHD), and cerebral degenerative disorders including Alzheimer's disease and other neurodegenerative and/or cognitive impairment diseases or disorders. In addition, sleep disorders notably refer to dyssomnias such as breathing-related sleep disorders including (obstructive or central) sleep apnea syndrome; periodic limb movement disorder (nocturnal myoclonus); restless leg syndrome; circadian rhythm sleep disorders including shift work sleep disorder; and time-zone-change (jet-lag) syndrome. Sleep disorders further refer to REM sleep interruptions. Parasomnias include arousal disorders and sleep-wake transition disorders; notably parasomnias include nightmare disorder, sleep terror disorder, and sleepwalking disorder. Sleep disorders associated with a general medical condition are in particular sleep disorders associated with diseases such as mental disorders, neurological disorders, neuropathic pain, and heart and lung diseases. Substance-induced sleep disorders include especially the subtypes insomnia type, parasomnia type and mixed type, and notably include conditions due to drugs which cause reductions in REM sleep as a side effect. Sleep disorders especially include all types of insomnias as defined before, as well as sleep-related dystonias; restless leg syndrome; sleep apneas; jet-lag syndrome; shift work sleep disorder, and delayed or advanced sleep phase syndrome. In addition, sleep disorders further include sleep disorders associated with aging.

Sleep disorders associated with a general medical condition include sleep disorders (especially insomnias) related to mental or neurologic disorders; notably sleep disorders (especially insomnias) associated with mood disorders (such as depressive disorders), epilepsy, autism spectrum disorders, attention deficit hyperactivity disorder (ADHD), and cerebral (neuro-)degenerative disorders including Alzheimer's disease and other neurodegenerative and/or cognitive impairment diseases or disorders; as well as sleep disorders (especially insomnias) associated with anxiety disorders, addiction disorders, or appetite disorders.

Mood disorders include major depressive episode, manic episode, mixed episode and hypomanic episode; depressive disorders including major depressive disorder, dysthymic disorders; bipolar disorders including bipolar I disorder, bipolar II disorder (recurrent major depressive episodes with hypomanic episodes), cyclothymic disorder; mood disorders including mood disorder due to a general medical condition (including the subtypes with depressive features, with major depressive-like episode, with manic features, and with mixed features), substance-induced mood disorder (including the subtypes with depressive features, with manic features, and with mixed features). Such mood disorders are especially major depressive episode, major depressive disorder, mood disorder due to a general medical condition; and substance-induced mood disorder.

Anxiety disorders can be distinguished by the primary object or specificity of threat, ranging from rather diffuse as in generalized anxiety disorder, to circumscribed as encountered in phobic anxieties (PHOBs) or post-traumatic stress disorders (PTSDs). Anxiety disorders may, thus, be defined as comprising generalized anxiety disorders (GAD), obsessive compulsive disorders (OCDs), acute stress disorders, posttraumatic stress disorders (PTSDs), panic anxiety disorders (PADs) including panic attacks, phobic anxieties (PHOBs), specific phobia, social phobia (social anxiety disorder), avoidance, somatoform disorders including hypochondriasis, separation anxiety disorder, anxiety disorders due to a general medical condition, and substance induced anxiety disorders. In a sub-embodiment, particular examples of circumscribed threat induced anxiety disorders are phobic anxieties or post-traumatic stress disorders. Anxiety disorders especially include post-traumatic stress disorders, obsessive compulsive disorders, panic attacks, phobic anxieties, and avoidance.

Addiction disorders may be defined as addictions to one or more rewarding stimuli, notably to one rewarding stimulus. Such rewarding stimuli may be of either natural or synthetic origin. Examples of such rewarding stimuli are substances/drugs {of either natural or synthetic origin; such as cocaine, amphetamines, opiates [of natural or (semi-)synthetic origin such as morphine or heroin], cannabis, ethanol, mescaline, nicotine, and the like}, which substances/drugs may be consumed alone or in combination; or other rewarding stimuli {of either natural origin (such as food, sweet, fat, or sex, and the like), or synthetic origin [such as gambling, or internet/IT (such as immoderate gaming, or inappropriate involvement in online social networking sites or blogging), and the like]}. In a sub-embodiment, addiction disorders relating to psychoactive substance use, abuse, seeking and reinstatement are defined as all types of psychological or physical addictions and their related tolerance and dependence components. Substance-related addiction disorders especially include substance use disorders such as substance dependence, substance craving and substance abuse;

substance-induced disorders such as substance intoxication, substance withdrawal, and substance-induced delirium. The expression “prevention or treatment of addictions” (i.e. preventive or curative treatment of patients who have been diagnosed as having an addiction, or as being at risk of developing addictions) refers to diminishing addictions, notably diminishing the onset of addictions, to weakening their maintenance, to facilitating withdrawal, to facilitating abstinence, or to attenuating, decreasing or preventing the occurrence of reinstatement of addiction (especially to diminishing the onset of addictions, to facilitating withdrawal, or to attenuating, decreasing or preventing the occurrence of reinstatement of addiction).

Appetite disorders comprise eating disorders and drinking disorders. Eating disorders may be defined as comprising eating disorders associated with excessive food intake and complications associated therewith; anorexias; compulsive eating disorders; obesity (due to any cause, whether genetic or environmental); obesity-related disorders including overeating and obesity observed in Type 2 (non-insulin-dependent) diabetes patients; bulimias including bulimia nervosa; cachexia; and binge eating disorder. Particular eating disorders comprise metabolic dysfunction; dysregulated appetite control; compulsive obesities; bulimia or anorexia nervosa. In a sub-embodiment, eating disorders may be defined as especially comprising anorexia nervosa, bulimia, cachexia, binge eating disorder, or compulsive obesities. Drinking disorders include polydipsias in psychiatric disorders and all other types of excessive fluid intake. Pathologically modified food intake may result from disturbed appetite (attraction or aversion for food); altered energy balance (intake vs. expenditure); disturbed perception of food quality (high fat or carbohydrates, high palatability); disturbed food availability (unrestricted diet or deprivation) or disrupted water balance.

The term “treatment of a sleep disorder” herein especially refers to the treatment of an insomnia; wherein in particular said treatment of a sleep disorder (especially insomnia) results in improvement on sleep onset/a decrease in latency to persistent sleep (LPS); and/or
an improvement on sleep maintenance/a decrease in wake time after sleep onset (WASO); and/or
an increase in total sleep time subjectively (sTST) assessed daily by the patient;

wherein it is understood that the above effects are measured from baseline and compared to placebo. Preferably, said treatment results, statistically significantly, in all of the above effects. More preferably, the treatment is not significantly associated with any treatment-emergent adverse events (TEAEs) (especially no severe TEAE), such TEAEs potentially including next-morning sleepiness effect (e.g. as assessed by a visual analog scale (VAS), e.g. every morning); rebound insomnia, withdrawal symptoms upon treatment discontinuation; or suicide, suicidal ideation, or self-injury.

The term “daytime clinical manifestations” as used herein refers to well known daytime symptoms of a sleep disorder, especially daytime clinical manifestations/symptoms of an insomnia, as specified in particular in the DSM-5.

For avoidance of any doubt, any method of treatment of a certain disease or disorder such as a sleep disorder, said method comprising administering daridorexant, or a pharmaceutically acceptable salt thereof, as set out in any one of embodiments 1) to 54) herein also discloses
daridorexant, or a pharmaceutically acceptable salt thereof, for use in the treatment of said disease or disorder as set out in any one of embodiments 1) to 54) herein,
the use of daridorexant, or of a pharmaceutically acceptable salt thereof, in the preparation of a medicament for the treatment of said disease or disorder as set out in any one of embodiments 1) to 54) herein;
daridorexant, or a pharmaceutically acceptable salt thereof, for use in such method of treatment of said disease or disorder as set out in any one of embodiments 1) to 54) herein;
a medicament for the treatment of said disease or disorder as set out in any one of embodiments 1) to 54) herein; and the like.

Likewise, if daridorexant, or a pharmaceutically acceptable salt thereof, is described as useful for the treatment of a certain disease or disorder such as a sleep disorder as set out herein, daridorexant, or a pharmaceutically acceptable salt thereof, is likewise suitable:
for use in the preparation of a medicament for the treatment of said disease or disorder as set out herein;
for use in a method to treat of said disease or disorder, said method comprising administering daridorexant, or a pharmaceutically acceptable salt thereof, as set out herein;
as a medicament for the treatment of said disease or disorder as set out herein; and the like.

Particular embodiments of the invention are described in the following examples, which serve to illustrate the invention in more detail without limiting its scope in any way.

### EXPERIMENTAL PART

### Abbreviations (as Used Hereinbefore or Hereinafter)

AE adverse event

DB double-blind

EOS End-of-Study

EOT End-of-Treatment

IDSIQ Insomnia Daytime Symptoms and Impacts Questionnaire

h Hour(s)

LPS latency to persistent sleep

min Minute(s)

PSG polysomnography

q.d. (quaque die): once daily (alternatively also qd)

sTST total sleep time subjectively

VAS visual analog scale

WASO wake time after sleep onset

### Example 1

The synthesis of daridorexant, [(S)-2-(5-Chloro-4-methyl-1H-benzoimidazol-2-yl)-2-methyl-pyrrolidin-1-yl]-(5-methoxy-2-[1,2,3]triazol-2-yl-phenyl)-methanone is described in WO2013/182972 and WO2015/083094. Crystalline salt forms of daridorexant are disclosed in WO2015/083071; and crystalline forms of daridorexant in free base form are disclosed in WO2015/083070

Daridorexant was used in the following clinical trial examples in form of the stable crystalline hydrochloric acid salt and can be prepared as described in WO2015/083071 and WO2018/202689.

Film-coated tablets for oral use containing daridorexant HCl of strenghth 10 mg, 25 mg and 50 mg (and corresponding placebo tablets) may be manufactured using conventional methods, e.g. using the following excipients:

Tablet core: mannitol, microcrystalline cellulose, povidone, croscarmellose sodium, silicon dioxide, magnesium stearate.

Film coat: hypromellose, microcrystalline cellulose, glycerine, talc, titanium dioxide, iron oxide.

### Example A): Multi-Center, Double-Blind, Randomized, Placebo-Controlled, Parallelgroup, Polysomnography Study to Assess the Efficacy and Safety of ACT-541468 in Adult and Elderly Subjects with Insomnia Disorder

The study comprises the following 3 phases: the screening phase, the treatment phase, and the safety follow-up phase. The screening phase starts with the signature of the informed consent form at Visit 1 and ends at Randomization (Visit 4), provided the subject fulfills all the eligibility criteria. It includes the Screening period and the Run-in period. The screening phase lasts 20 to 31 days. The Screening period starts with Visit 1 and ends at Visit 2. During the Screening period, the investigator verifies eligibility criteria and eligible subjects perform a one-night polysomnography (PSG) assessment. The Screening period lasts 7 to 18 days to allow time to perform all required procedures at Visit 1, the PSG assessment and collect the minimum number of eDiary entries (i.e., 7 days) between Visit 1 and Visit 2. The Run-in period starts with Visit 2 and ends at Randomization (i.e., Visit 4). At Visit 2 eligible subjects are allocated a single-blinded placebo treatment that is taken daily. During the Run-in period subjects come to the site for Visit 3, which consists of 2 PSG nights and is performed when the subject has completed the eDiary for at least 7 days and eligibility is confirmed. The Run-in period lasts 13 to 24 days, to allow collection of the minimum number of eDiary entries (i.e., 7 days), perform 2 PSG nights at Visit 3, and receive the eligibility confirmation from the PSG central reader. The double-blind (DB) treatment phase lasts 3 months. It starts at Randomization (Visit 4). DB study treatment is taken daily. A safety telephone call is performed at Visit 5 to collect information about adverse events (AEs) and concomitant medications. Sleep parameters of each subject are objectively assessed with 2 consecutive PSG nights at Visit 6 and Visit 8. A safety visit without PSG night is performed at Visit 7. An eDiary is completed every day during the treatment phase. End-of-Double-Blind-Treatment (EODBT) is reached in the second morning of Visit 8. The safety follow-up phase starts after EODBT. It consists of a single-blind placebo run-out period of 7 days and a safety follow-up period. The Run-out period starts in the evening of Visit 9. Visit 9 consists of one PSG night on single-blind placebo treatment. Visit 9 is followed by 6 days at home with single-blind placebo treatment. The eDiary is completed every day during the Run-out period. The end of the Run-out period (End-of-Treatment [EOT]) is reached after all visit assessments have been performed at Visit 10. The Safety follow-up period starts after EOT and ends 30 days after the last dose of DB study treatment intake for subjects that are not enrolled in the ID-078A303 extension study. Subjects who complete DB study treatment and the Run-out period are eligible to enter the ID-078A303 extension study (if approved by the national health authorities and local Independent Ethics Committees/Institutional Review Boards). For these subjects, the safety follow-up period ends on the date of enrolment into ID-078A303. End-of-Study (EOS) for an individual subject is defined as the date of the 30-day follow-up telephone call (Visit 11) or the date of enrolment into the ID-078A303 extension study. If a subject is prematurely discontinued from study treatment EOS is performed as planned on Day 115. If a subject withdraws consent and does not wish to participate in the study any longer, EOS is the date of consent withdrawal for this subject. If a subject is declared lost to follow-up, EOS is the date of last successful contact for this subject.

Study Treatments

Investigational treatment: ACT-541468 tablets at strengths of 25 mg and 50 mg is administered orally, once daily in the evening during the DB treatment period.

Placebo: ACT-541468-matching placebo is administered orally, once daily in the evening during the single-blind run-in period, the DB treatment period and the single-blind run-out period.

Endpoints

Primary Efficacy Endpoints

The primary efficacy endpoints of this study are defined as:
the change from baseline to Month 1 in WASO (sleep maintenance)
the change from baseline to Month 3 in WASO
the change from baseline to Month 1 in LPS (sleep onset)
the change from baseline to Month 3 in LPS

Baseline is defined as mean of the 2 PSG nights at Visit 3. Month 1 and Month 3 are defined as the mean of the 2 PSG nights at Visit 6 and Visit 8, respectively.

LPS (min) is the time from start of recording to the beginning of the first continuous 20 epochs (i.e., 10 min) scored as non-awake, i.e., epochs scored as either sleep stage 1 (S1), sleep stage 2 (S2), sleep stage 3 (slow wave sleep) or REM, as determined by PSG.

WASO is the time (min) spent awake after onset of persistent sleep until lights on, as determined by PSG.

Secondary Efficacy Endpoints

The secondary efficacy endpoints of this study are defined as:
the change from baseline to Month 1 in sTST.
the change from baseline to Month 3 in sTST.
the change from baseline to Month 1 in Insomnia Daytime Symptoms and Impacts Questionnaire (IDSIQ) sleepiness domain score.
the change from baseline to Month 3 in IDSIQ sleepiness domain score.
Baseline is the mean value based on the screening sleep diary/IDSIQ entries performed at home in the 7 days immediately preceding the first PSG at Visit 3.
‘Month 1’ is the mean value based on the sleep diary/IDSIQ entries performed at home in the 7 days immediately preceding the first PSG at Visit 6.
‘Month 3’ is the mean value based on the sleep diary/IDSIQ entries performed at home in the 7 days immediately preceding the first PSG at Visit 8.

Safety Endpoints:

In addition to the standardized collection of AEs, safety data specific to insomnia and its treatment is assessed as follows:
Withdrawal effects (physical dependence) upon treatment discontinuation is assessed based on the changes from last assessment on DB treatment (Visit 8, 2nd morning) to run-out period in the Benzodiazepine Withdrawal Symptom Questionnaire (BWSQ) total score (Visit 9 and Visit 10), the occurrence of relevant AEs and marked ECG abnormalities.
Rebound insomnia is assessed based on objective sleep parameters (WASO, LPS, and TST) at Visit 9 as compared to Visit 3. It is also assessed using subjective sleep parameters (subjective WASO [sWASO], subjective Latency to Sleep Onset [sLSO], and sTST) from run-out period as compared to baseline.
Next-day residual effects is assessed based on changes from baseline (Visit 3) to Month 1 and Month 3 in:
   Coding sub-test©
   Sheehan Disability Scale© (SDS©)
   Scores on the visual analog scale (VAS; mm)
Serious adverse events up to 30 days after DB study treatment discontinuation or until enrollment into the extension study.
Treatment-emergent AEs (TEAEs) up to 30 days after DB study treatment discontinuation or until enrollment into the extension study.
AEs leading to premature discontinuation of the DB study treatment.
AEs of special interest (AESIs) after adjudication by an Independent Safety Board (ISB):
   narcolepsy-like symptoms (i.e., excessive daytime sleepiness [EDS], cataplexy and complex sleep behavior events including hallucinations/sleep paralysis)
   suicide/self-injury.
Change from baseline (Visit 3) to Month 1 (Visit 6) and Month 3 (Visit 8) in vital signs (mean of the 2 PSG nights in systolic and diastolic blood pressure [BP] and pulse rate).
Change from baseline (Visit 1) to Month 3 (Visit 8) in body weight.
Marked ECG abnormalities on DB study treatment.
Change from baseline (Visit 3) to Month 3 (Visit 8) and the end of run-out (Visit 10) in ECG variables.
Marked laboratory abnormalities on DB study treatment.
Change from baseline (Visit 3) to Month 1 (Visit 6) and Month 3 (Visit 8) in laboratory variables.
Occurrence of suicidal ideation and/or behavior on DB study treatment based on C-SSRS©.

Statistical Methodology

Analysis of the Primary and Secondary Efficacy Endpoints:

The Type I error rate is controlled for the testing of multiple null hypotheses associated with the two primary endpoints (LPS and WASO) and two other endpoints (sTST and IDSIQ) assessed at 1 and 3 months of treatment, and the two dose levels included in this study, i.e., 25 mg and 50 mg.

The eight statistical null hypotheses associated with the primary efficacy endpoints are:

Sleep Maintenance:
H1_{WASO}: Higher Dose-Placebo=0 for WASO at Month 1
H2_{WASO}: Higher Dose-Placebo=0 for WASO at Month 3
H3_{WASO}: Lower Dose-Placebo=0 for WASO at Month 1
H4_{WASO}: Lower Dose-Placebo=0 for WASO at Month 3

Sleep Onset:
H1_{LPS}: Higher Dose-Placebo=0 for LPS at Month 1
H2_{LPS}: Higher Dose-Placebo=0 for LPS at Month 3
H3_{LPS}: Lower Dose-Placebo=0 for LPS at Month 1
H4_{LPS}: Lower Dose-Placebo=0 for LPS at Month 3
   The eight statistical null hypotheses associated with the secondary efficacy endpoints are: Sleep quantity:
H1_{sTST}: Higher Dose-Placebo=0 for sTST at Month 1
H2_{sTST}: Higher Dose-Placebo=0 for sTST at Month 3
H3_{sTST}: Lower Dose-Placebo=0 for sTST at Month 1
H4_{sTST}: Lower Dose-Placebo=0 for sTST at Month 3

Next-Day Performance:
H1_{IDSIQ}: Higher Dose-Placebo=0 for IDSIQ sleepiness domain score at Month 1
H2_{IDSIQ}: Higher Dose-Placebo=0 for IDSIQ sleepiness domain score at Month 3
H3_{IDSIQ}: Lower Dose-Placebo=0 for IDSIQ sleepiness domain score at Month 1
H4_{IDSIQ}: Lower Dose-Placebo=0 for IDSIQ sleepiness domain score at Month 3

where ‘Higher Dose’, ‘Lower Dose’, and ‘Placebo’ represent the mean change from baseline for the given endpoint (WASO, LPS, sTST or IDSIQ sleepiness domain score) and time point (Month 1 or Month 3) for the 50 mg, 25 mg, and placebo treatment group, respectively.

Each null hypothesis is tested against the alternative hypothesis: that ACT-541468 improves WASO/LPS/sTST/IDSIQ sleepiness domain score at the given dose (25 or 50 mg) and time point (Month 1 or Month 3) compared to placebo.

Insomnia Daytime Symptoms and Impacts Questionnaire (IDSIQ)

The IDSIQ patient reported outcome (PRO) instrument (S. Hudgens et al., The Patient−Patient-Centered Outcomes Research (2021) 14:249-268) is programmed on the electronic hand-held device in the subject's language, and must be completed every day in the evening before the evening sleep diary by the subject without study staff input or interference from Screening (Visit 1) until EOT (Visit 10). The IDSIQ is structured in 3 domains (i.e., alertness/cognition; negative mood; tiredness/sleepiness) and contains overall 14 items, each based on an 11-point numeric rating scale. For each domain the total rating score of all associated items is considered.

| Item Concept | | Domain Concept | | Overall Concept |
|---|---|---|---|---|
| Item 1. How clear-headed did you feel today? | | | | |
| Item 2 How well were you able to concentrate today? | | | | |
| Item 3 How forgetful did you feel today? | | Alert/Cognition | | |
| Item 9 How much of an effort was it to perform daily activities | {close oversize brace} | Domain | | |
| (i.e. reading, cleaning, work, school) today? | | | | |
| Item 10. How refreshed did you feel today? | | | | Insomnia |
| Item 14. How awake did you feel today? | | | | Daytime |
| Item 4. How worried did you feel today? | | | {close oversize brace} | Symptoms and |
| Item 5 How frustrated by your lack of sleep did you feel today? | | Mood | | Impacts |
| Item 6. How irritable did you feel today? | {close oversize brace} | Domain | | Questionnaire |
| Item 7. How stressed did you feel today? | | | | (IDSIQ) |
| Item 8 How energetic did you feel today? | | | | |
| Item 11. How mentally tired did you feel today? | | Sleepiness | | |
| Item 12. How physically tired did you feel today? | {close oversize brace} | Domain | | |
| Item 13. How sleepy did you feel today? | | | | |

This tool is based on an existing instrument, the Daytime Insomnia Symptom Scale [Buysse et al., Sleep Med. 2007 April; 8(3):198-208]. The psychometric validation of the IDSIQ instrument was performed in a Phase 2 study (NCT03056053; ID-078A203) conducted in subjects with insomnia. The instrument was validated according to FDA guidelines.

Results:

The study showed statistically significant effects on all primary endpoints and the secondary efficacy endpoint sTST, for both the 25 mg and the 50 mg strength. Thus, daridorexant improved sleep onset and sleep maintenance, with significant reductions in WASO and LPS at both doses; and increased the subjective total sleep time (sTST) significantly at both doses.

For the secondary endpoint IDSIQ sleepiness domain score the results are summarized below: The sleepiness domain of the IDSIQ (domain of items 8, 11, 12, 13 as described above) was significantly improved by daridorexant at the 50 mg dose at month 1 and month 3, and a numerical trend was observed at the 25 mg dose at both time points.

The IDSIQ total score and the alert/cognition and mood domain scores also decreased with both doses of daridorexant at all timepoints versus placebo. Daridorexant 50 mg improved IDSIQ mood and alert/cognition domains (reduced scores) and total score at both timepoints, with p-values of comparisons versus placebo (not adjusted for multiplicity) all 0.0005.

Improvements in sleep were maintained over 3 months and were associated with a gradual improvement in daytime functioning over time.

Daridorexant was well tolerated and had a favorable safety profile in both adult and elderly patients. The rate of adverse events was comparable between placebo and daridorexant at both treatment doses. The absence of next-morning sleepiness is consistent with the pharmacokinetic profile of daridorexant. The incidence of somnolence was low (lower with daridorexant 50 mg versus placebo) and not dose dependent. The most frequent treatment-emergent adverse events (TEAEs) were nasophyringitis and headache. The number of serious adverse events were higher in the placebo group compared to the daridorexant treatment groups. There was no next-morning sleepiness effect assessed by a visual analog scale (VAS) every morning. There was no rebound insomnia, or withdrawal symptoms upon discontinuation, and no suicide, suicidal ideation, or self-injury were observed. Fewer falls in the 50 mg dose group compared to placebo were observed in the trial of Example A).

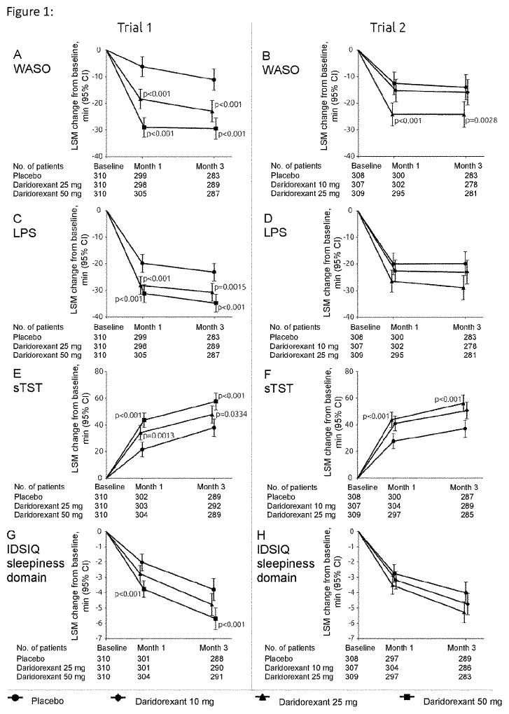

FIG. 1: Primary and Secondary Efficacy End Points:

The least squares mean change from baseline to Months 1 and 3 for WASO (Panels A and B), LPS (Panels C and D), sTST (Panels E and F), and IDSIQ sleepiness domain score (Panel G and H) in Trial 1 (Example A; left hand side) and Trial 2 (Example B; right hand side) are shown. WASO and LPS data are the mean of polysomnography recordings over two consecutive nights during the 3-month double-blind treatment period. Data for sTST and IDSIQ sleepiness domain score are based on the mean of daily entries in the seven days prior to polysomnography nights. Error bars represent the 95% confidence interval. The two-sided p-values shown are versus placebo and are statistically significant under type I error control.

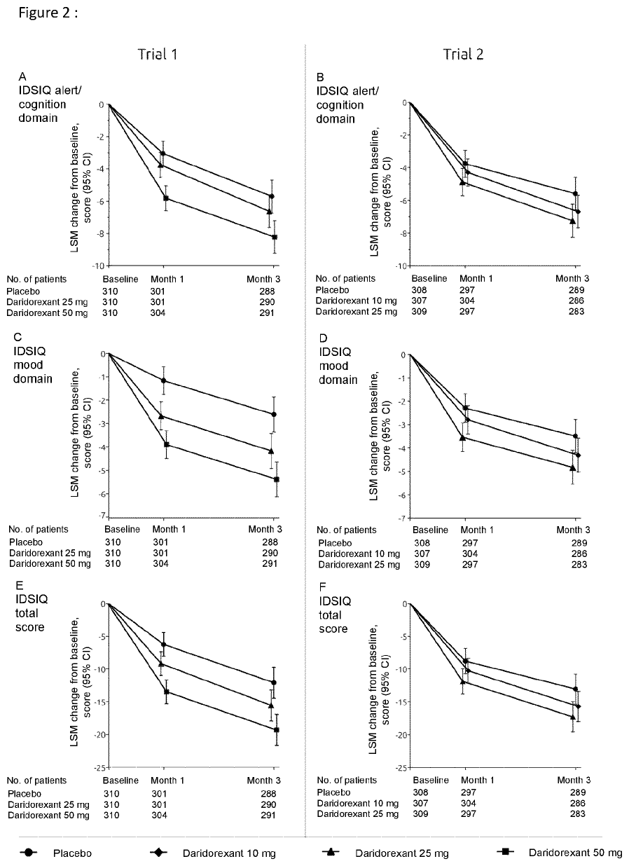

FIG. 2: Other IDSIQ End Points:

The least squares mean change in score from baseline to Months 1 and 3 for the alert/cognition domain (Panels A and B), mood domain (Panels C and D), and total score (Panels E and F) of the Insomnia Daytime Symptoms and Impacts Questionnaire (IDSIQ) in Trial 1 (Example A; left hand side) and Trial 2 (Example B; right hand side) are presented. IDSIQ scores are based on the mean of daily entries in the seven days prior to polysomnography nights. Error bars represent the 95% confidence interval.

### Example B): Multi-Center, Double-Blind, Randomized, Placebo-Controlled, Parallelgroup, Polysomnography Study to Assess the Efficacy and Safety of ACT-541468 in Adult and Elderly Subjects with Insomnia Disorder

The study (NCT03575104) is conducted in analogy to the study of Example A), using daridorexant strengths of 10 mg and 25 mg.

Results:

See FIG. 1 and FIG. 2.

Daridorexant 25 mg, but not 10 mg, significantly decreased WASO and increased sTST at month 1 and month 3 versus placebo, while the decrease in LPS did not reach statistical significance. After log-transformation, daridorexant 25 mg significantly reduced LPS at month 1 and month 3. With regard to the sleepiness domain of the IDSIQ a numerical trend was observed at the 25 mg dose at both time points.

### Example C): Multi-Center, Double-Blind, Parallel-Group, Randomized, Placebo-Controlled, Three Doses, 40-Week Extension to Studies ID-078A301 and ID-078A302 to Assess the Long Term Safety and Tolerability of ACT-541468 in Adult and Elderly Subjects with Insomnia Disorder

The study (NCT03679884) is an extension study of the studies of Example A) and Example B) and may provide longer-term data, especially in confirmation of effects observed in Example A) and/or B) at the month 3 time-point.

1. A method of treating a sleep disorder; said method comprising administering to a subject in need thereof an effective amount of daridorexant: 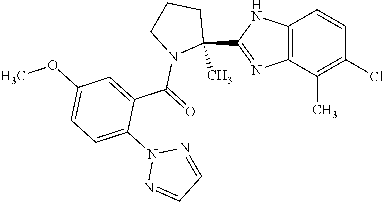 [SMILES: COc1ccc(-n2nccn2)c(C(=O)N2CCC[C@@]2(C)c2nc3c(C)c(Cl)ccc3[nH]2)c1]
in free or pharmaceutically acceptable salt form;
wherein daridorexant improves the daytime performance of said subject.

2. The method according to claim 1, wherein said improvement of daytime performance of said subject is assessed by at least one of:
the IDSIQ daytime alert/cognition domain score; and/or
the IDSIQ daytime mood domain score, and/or
the IDSIQ daytime sleepiness domain score.

3. A method of treating a sleep disorder; said method comprising administering to a subject in need thereof an effective amount of daridorexant in free or pharmaceutically acceptable salt form; wherein daridorexant reduces daytime clinical manifestations associated with said sleep disorder.

4. The method according to claim 3, wherein said daytime clinical manifestations associated with said sleep disorder are symptoms of daytime sleepiness, especially as assessed by the IDSIQ daytime sleepiness domain score.

5. The method according to claim 1, wherein said improvement of daytime performance is expressed by subjects to whom daridorexant has been administered, wherein said subjects feel, cumulatively, less physically tired, less mentally tired, less sleepy and more energetic during the day.

6. The method according to claim 1, wherein said sleep disorder is a dyssomnia or a sleep disorder associated with a general medical condition.

7. The method according to claim 1, wherein said sleep disorder is an insomnia.

8. The method according to claim 1, wherein said treatment of said sleep disorder results in at least one, preferably all of the following treatment effects:
a decrease in latency to persistent sleep (LPS); and/or
a decrease in wake time after sleep onset (WASO); and/or
an increase in total sleep time subjectively (sTST) assessed daily by the patient.

9. The method according to claim 1, wherein said subject has been diagnosed as having difficulties with sleep onset and/or sleep maintenance.

10. The method according to claim 1, wherein daridorexant in free or pharmaceutically acceptable salt form, is administered in a unit dose of about 25 mg of daridorexant, or in a unit dose of about 50 mg of daridorexant.

11. The method according to claim 1, wherein the daridorexant is in hydrochloric acid salt form.

12. The method according to claim 1, wherein daridorexant in free or pharmaceutically acceptable salt form is administered in form of a tablet; wherein said tablet is a film coated tablet comprising
a tablet core; said tablet core comprising at least two, preferably all of the following excipients: mannitol, microcrystalline cellulose, povidone, croscarmellose sodium, silicon dioxide, and/or magnesium stearate; and
a film coat; said film coat comprising at least two, preferably all of the following excipients: hypromellose, microcrystalline cellulose, glycerine, talc, titanium dioxide, and/or iron oxide.

13. The method according to claim 1, wherein daridorexant in free or pharmaceutically acceptable salt form is administered once daily within 1 h before bedtime, in particular within 30 min before bedtime.

14. The method according to claim 1, wherein said sleep disorder is a chronic sleep disorder.

15. A method of treating daytime sleepiness in a subject, comprising administering to said subject an effective amount of daridorexant in free or pharmaceutically acceptable salt form, wherein said subject has a reduction in daytime sleepiness as assessed by the IDSIQ daytime sleepiness domain score; wherein especially said subject has been diagnosed as having a sleep disorder.

16. A method of treating insomnia to improve sleep and daytime functioning, comprising administering to a subject in need thereof an effective amount of daridorexant in free or pharmaceutically acceptable salt form, wherein notably said subject has a reduction in daytime sleepiness as assessed by the IDSIQ daytime sleepiness domain score.

17. (canceled)

18. The method according to claim 3, wherein said reduction of daytime clinical manifestations associated with said sleep disorder is expressed by subjects to whom daridorexant has been administered, wherein said subjects feel, cumulatively, less physically tired, less mentally tired, less sleepy and more energetic during the day.

19. The method according to claim 3, wherein said sleep disorder is a dyssomnia or a sleep disorder associated with a general medical condition.

20. The method according to claim 3, wherein said sleep disorder is an insomnia.

21. The method according to claim 3, wherein said treatment of said sleep disorder results in at least one, preferably all of the following treatment effects:
a decrease in latency to persistent sleep (LPS); and/or
a decrease in wake time after sleep onset (WASO); and/or
an increase in total sleep time subjectively (sTST) assessed daily by the patient.

22. The method according to claim 3, wherein said subject has been diagnosed as having difficulties with sleep onset and/or sleep maintenance.

23. The method according to claim 3, wherein daridorexant in free or pharmaceutically acceptable salt form, is administered in a unit dose of about 25 mg of daridorexant, or in a unit dose of about 50 mg of daridorexant.

24. The method according to claim 3, wherein the daridorexant is in hydrochloric acid salt form.

25. The method according to claim 3, wherein daridorexant in free or pharmaceutically acceptable salt form is administered in form of a tablet; wherein said tablet is a film coated tablet comprising
a tablet core; said tablet core comprising at least two, preferably all of the following excipients: mannitol, microcrystalline cellulose, povidone, croscarmellose sodium, silicon dioxide, and/or magnesium stearate; and
a film coat; said film coat comprising at least two, preferably all of the following excipients: hypromellose, microcrystalline cellulose, glycerine, talc, titanium dioxide, and/or iron oxide.

26. The method according to claim 3, wherein daridorexant in free or pharmaceutically acceptable salt form is administered once daily within 1 h before bedtime, in particular within 30 min before bedtime.

27. The method according to claim 3, wherein said sleep disorder is a chronic sleep disorder.
